(19) United States
(12) Patent Application Publication
(10) Pub. No.: US 2020/0086092 A1
(43) Pub. Date: Mar. 19, 2020

(54) GUIDEWIRE RETAINERS FOR USE WITH CATHETERS AND RELATED METHODS AND KITS

(71) Applicant: Duke University, Durham, NC (US)

(72) Inventors: Nathan Waldron, Durham, NC (US); Mark Stafford-Smith, Durham, NC (US)

(21) Appl. No.: 16/617,693

(22) PCT Filed: Jun. 1, 2018
(86) PCT No.: PCT/US2018/035525

§ 371 Date: Nov. 27, 2019

Related U.S. Application Data
(60) Provisional application No. 62/513,565, filed on Jun. 1, 2017.

(51) Int. Cl. A61M 25/09; A61M 25/06

(52) U.S. Cl. CPC A61M 25/09041; A61M 2025/09125; A61M 25/065

(57) ABSTRACT

Guidewire retainers for use with catheters and related methods and kits are disclosed. According to an aspect, a guidewire retainer includes a body that defines an interior space therein and an opening at one end that provides passage from an exterior of the body to the interior space. The interior space can be shaped and sized for holding an end of a guidewire. The guidewire retainer also includes a locking mechanism configured to close the body for attaching the body to the end of the guidewire.

### CROSS REFERENCE TO RELATED APPLICATION

This application claims priority to U.S. Provisional Patent Application No. 62/513,565, filed Jun. 1, 2017, and titled GUIDEWIRE-KEEPING DEVICE AND METHODS OF MAKING AND USING SAME, the disclosure of which is incorporated herein by reference in its entirety.

### TECHNICAL FIELD

The presently disclosed subject matter relates to medical devices and instruments. More particularly, the presently disclosed subject matter relates to guidewire retainers for use with catheters and related methods and kits.

### BACKGROUND

Guidewires are often used for various medical procedures in the treatment of the human body. In many instances, guidewires are used to guide a catheter to a site within a body cavity, duct, or vessel. For example, catheter placement, or catheterization, may be within a large vein, such as a vein in the neck, chest, groin, or veins in the arms. Catheters placed in these areas may be used to administer medication or fluids that are unable to be ingested by the mouth or may harm a smaller peripheral vein.

Catheterization of central veins is a routine technique that is widely used in various settings, such as operating rooms, emergency departments, and intensive care units (ICUs). Seldinger's technique is commonly used to place central venous and arterial catheters. However, this technique and others have several potentially serious risks. For example, a guidewire may be lost during central venous catheter (CVC) insertion. In the instance of guidewire loss within a patient, an additional medical procedure may be required, such as fluoroscopy-guided endovascular snaring, for retrieval of the guidewire. Additionally, sometimes the loss of a guidewire during catheterization is not recognized and subsequently leads to other serious and even occasionally fatal complications. For at least these reasons, there is a need for improved systems and techniques for preventing the loss of guidewires during a medical procedure.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Disclosed herein are guidewire retainers for use with catheters and related methods and kits. According to an aspect, a guidewire retainer includes a body that defines an interior space therein and an opening at one end that provides passage from an exterior of the body to the interior space. The interior space can be shaped and sized for holding an end of a guidewire. The guidewire retainer also includes a locking mechanism configured to close the body for attaching the body to the end of the guidewire.

According to another aspect, a method of preventing a guidewire from completely entering a tubular object, such as a catheter or dilator, includes providing a guidewire retainer. The guidewire retainer includes a body that defines an interior space therein and an opening at one end that provides passage from an exterior of the body to the interior space. The interior space can be shaped and sized for holding an end of a guidewire. The guidewire retainer also includes a locking mechanism configured to close the body for attaching the body to the end of the guidewire.

According to another aspect, a kit includes a tubular object or guidewire. The kit also includes a guidewire retainer including a body that defines an interior space therein and an opening at one end that provides passage from an exterior of the body to the interior space. The interior space can be shaped and sized for holding an end of a guidewire. The guidewire retainer also includes a locking mechanism configured to close the body for attaching the body to the end of the guidewire.

### BRIEF DESCRIPTION OF THE DRAWINGS

The foregoing summary, as well as the following detailed description of various embodiments, is better understood when read in conjunction with the appended drawings. For the purposes of illustration, there is shown in the drawings exemplary embodiments; however, the presently disclosed subject matter is not limited to the specific methods and instrumentalities disclosed. A brief description of the drawings follows.

FIG. 1 is a perspective view of a catheter, guidewire, and guidewire retainer assembled in accordance with embodiments of the present disclosure;

FIG. 2 is a perspective view of an example wireguide retainer in accordance with embodiments of the present disclosure;

FIG. 3 is a perspective view of the guidewire retainer shown in FIG. 2 in a closed position; and

FIG. 4 is a perspective view of the guidewire retainer shown in FIG. 2 in a closed position with a guidewire 102 inserted therein.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

As used herein, the term “user” and “subject,” are used interchangeably herein and refer to an individual (e.g., human), such as a medical doctor, performing a surgical procedure and using a catheter. A patient or another subject may be the individual having the surgical procedure.

As used herein, the term “catheter” is used herein to refer to any suitable tube utilized for insertion in a subject's body to treat diseases or to perform a surgical procedure. Catheters are generally tubular in shape and can have any suitable size depending on the specific surgical procedure of use. During a surgical procedure, a catheter may be inserted into a body cavity, duct, or vessel. They may allow drainage, administration of fluids or gases, access by surgical instruments or guidewires, and also perform a wide variety of other tasks depending on the type of catheter. Commonly, catheters are thin and flexible, although they can be available in varying levels of stiffness depending on the surgical procedure application. The catheter insertion process is often referred to as “catheterization”.

As referred to herein, the term “guidewire” is used herein to refer a wire or device that fits inside a catheter to assist in inserting, positioning, and moving a catheter into or through an area within a subject's body. For example, a distal end of a guidewire may be advanced into the catheter such that it extend from the distal end of the catheter. The guidewire may subsequently be manipulated and advanced through and into a body cavity, duct, or vessel to assist with moving the catheter into a desired position. The guidewire may be removed once the catheter is in place. Guidewires may be characterized by their pushability, steerability, torque, and opacity (i.e., level of visibility under fluoroscopic imaging). Guidewires typically come in one of two configurations: solid steel or nitinol core wires and solid core wires wrapped in a smaller wire coil or braid. Some wires are coated with a polymer, such as silicone or polytetrafluoroethylene (PTFE) to increase lubricity. Example guidewire diameters include, but are not limited to, the range of between 0.014 and 0.038 inches. Example guidewire lengths include, but are not limited to, the range of 80 and 450 centimeters.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any nonclaimed element as essential to the practice of the presently disclosed subject matter.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

Unless otherwise defined, all technical terms used herein have the same meaning as commonly understood by one of ordinary skill in the art to which this disclosure belongs.

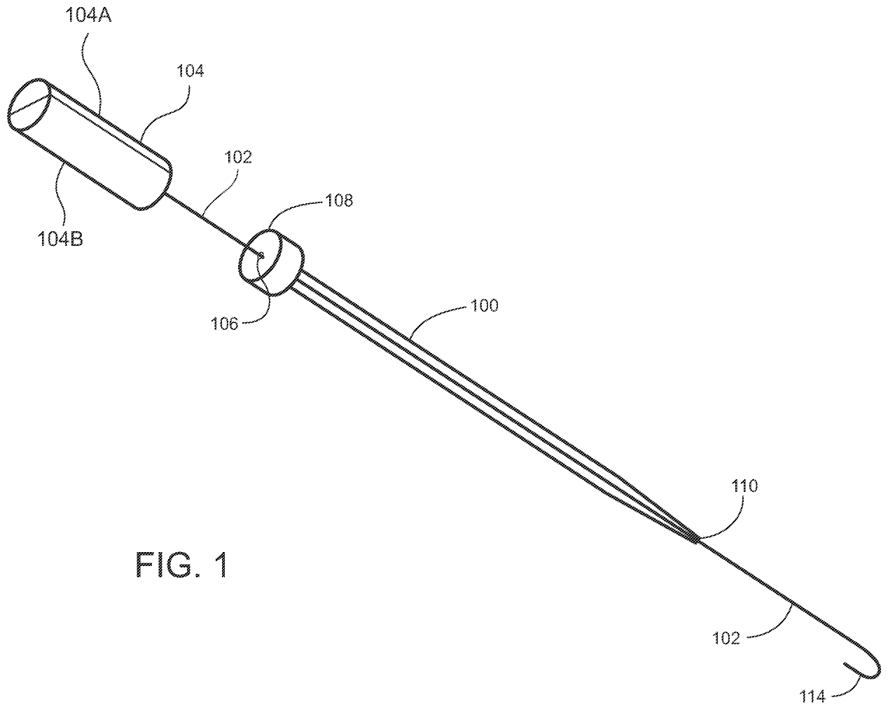

FIG. 1 illustrates a perspective view of a catheter 100, guidewire 102, and guidewire retainer 104 assembled in accordance with embodiments of the present disclosure. Referring to FIG. 1, the catheter 100 may be any suitable type of catheter having an interior passageway through which the guidewire 102 can pass between an opening 106 at a proximal end 108 and an opening at a distal end 110. In this example, the guidewire 102 is a “J” tip guidewire as characterized by the “J” shape at a distal end 114 of the guidewire 102. The guidewire 102 may be utilized for insertion and placement of the catheter 100 into an interior of a patient's body.

The guidewire retainer 104 is attached to a proximal end (not shown) of the guidewire 102. The guidewire retainer 104 may be attached to the proximal end of the guidewire 102 by any suitable technique, such as by a clamping force. For example, the guidewire retainer 104 may include parts 104A and 104 that can attach together and apply a compression force therebetween for clamping onto the proximal end of the guidewire 102. The compression force may be sufficient such that an operator of the guidewire 102 and catheter cannot easily remove or loosen the guidewire retainer 104 from the guidewire 102 during a surgical procedure.

The guidewire retainer 104 is designed to prevent the proximal end of the guidewire 102 from entering into the catheter through the opening 106. More particularly, the guidewire retainer is sized such that it cannot fit through the opening 106. Thereby, the proximal end of the guidewire 102 is prevented from traversing through the catheter 100 such that the guidewire 102 could become lost during a procedure. For example, upon removal of the catheter 100 from the patient's body, the guidewire 102 would necessarily also be removed due to the guidewire retainer 104 preventing the proximal end of the guidewire 102 from entering the opening 106.

In the example of FIG. 1, the guidewire retainer 104 is shown as being cylindrical in shape. It should be understood that the guidewire retainer 104 may alternatively be any other suitable shape such as spherical, conical, cubical, rectangularly, square, or rhomboidal.

The guidewire retainer 104 may be attached to the guidewire 102 by any suitable technique. For example, the guidewire retainer 104 may include a locking mechanism that, when pressed, locks the guidewire retainer 104 to the guidewire. Also, in this example, the locking mechanism may be suitably depressed to unlock the guidewire retainer 104 from the guidewire.

In accordance with embodiments, the guidewire retainer 104 may be configured for attachment to any suitable gauge of guidewire. For example, the guidewire retainer 104 may be configured to attach to a 20 gauge wire that has a length of about 1 to about 3 centimeters and a width of about 0.1 to about 1 centimeter. In other examples, the guidewire retainer 104 may be configured to attach to a guidewire of about 14 gauge to about 28 gauge. In some embodiments, the guidewire retainer 104 may have a length of about 1 to 2 centimeters and a width of about 0.4 to about 0.6 centimeters.

In accordance with embodiments, a guidewire may be prevented from completely entering a catheter by use of a guidewire retainer as disclosed herein. For example, referring to the example of FIG. 1, the distal end 114 of the guidewire 102 may be inserted through the skin of a patient and into a vessel through a needle. Subsequently, the needle may be removed. A dilator and/or catheter may be slid over the guidewire for insertion into the skin of the patient. Subsequently, the guidewire retainer 104 may be attached to the proximal end of the guidewire 102 as shown in FIG. 1 to thereby prevent the guidewire 102 from completely entering the catheter or dilator. In an example, the guidewire retainer 104 may be detached from the guidewire 102 once the procedure is complete.

A guidewire retainer and associated components of the present disclosure may be made of any suitable material, such as plastics and metals, such as stainless steel. In some embodiments, the material comprises a biocompatible material suitable for medical use. In certain embodiments, the material, or combination of materials, meet the requirements as outlined above. Examples include, but are not limited to, (a) polyurethanes, including but not limited to, polycarbonate-based polyurethanes, polyether-based polyurethanes (e.g., aliphatic, aromatic, etc.), and thermoplastic polyurethanes (e.g., polyvinyl chloride (PVC), etc.); (b) polyamides; (c) fluoropolymers, including but not limited to, polytetrafluoroethylene (PTFE), FEP, ETFE, PFA and MFA; (d) polyolefins (e.g., high density polyethylene, etc.); (e) polyimides; (f) thermoplastic polymers, such as polyaryletherketone (PAEK) and polyether ether ketone (PEEK); (g) polycarbonate; (h) polycarbonate urethane; (i) silicone; (j) acrylic compounds; (k) thermoplastic polyesters; (l) polypropylene; (m) low-density polyethylenes; (n) nylon; (o) sulfone resins; (p) high density polyethylenes; (q) silicone, and silicone-based materials and (r) other synthetic biocompatible polymers; and combinations thereof. Further, the guidewire-keeping device according to the present disclosure may be manufactured by any number of methods, including, but not limited to, injection molding, 3D-printing, and the like.

In accordance with embodiments, a kit may be provided that includes a catheter and/or dilator, a guidewire, a guidewire retainer according to the present disclosure, and instructions for use.

In an example use case, a central venous line procedure may involve a 20 G J-shaped guidewire. During such a procedure, the guidewire is initially inserted through the skin of a subject and into the vessel through a needle, the needle then being removed and a dilator and/or catheter is/are then threaded over the guidewire for insertion into the skin. The guidewire retainer according to the present disclosure is then attached to the proximal end of the guidewire such that, with manipulation of the catheter and/or dilator, the guidewire cannot slip unobserved inside the catheter/dilator to be inadvertently inserted into the subject.

Yet another aspect of the present disclosure provides a kit. The kit can include a catheter and/or dilator, a guidewire retainer according to the present disclosure, a guidewire, and instructions for use.

Example uses of the presently disclosed catheters, guidewires, and guidewire retainers include, but are not limited to, Seldinger's technique, placement of any percutaneous drainage catheter (e.g., “pigtail” chest tubes, percutaneous abdominal or biliary drains, placement of abdominal drains), and the like.

A guidewire retainer in accordance with embodiments of the present disclosure may be provided in a kit along with one or more catheters and/or one or more guidewires for use therewith. In an example, a guidewire retainer may be provided in a central line kit.

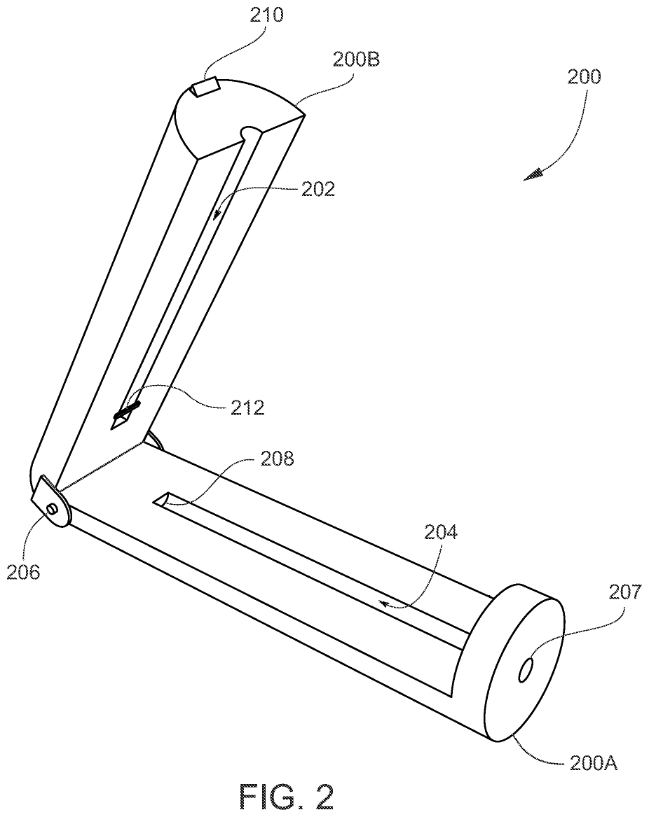

FIG. 2 illustrates a perspective view of an example wireguide retainer 200 in accordance with embodiments of the present disclosure. Referring to FIG. 2, the wireguide retainer 200 includes a bottom portion 200A and a top portion 200B that can, in a closed state, attach together and apply a compression force there between for clamping onto a proximal end of a guidewire (not shown). When in the closed state, the portions 200A and 200B can define a body and an interior space therein that is shaped and sized to hold the proximal end of the guidewire. The wireguide retainer 200 is shown in FIG. 2 in an open state such that grooves 202 and 204 are revealed. When in the closed state, the grooves 202 and 204 join to form the interior space for holding the proximal end of the guidewire.

In this example, portions 200A and 200B are pivotally attached to one another. The portions 200A and 200B may pivot about a pivot 206 to move between the open and closed states. Alternatively, the portions 200A and 200B may be attached in another suitable manner to form an interior space to hold the guidewire.

At one end of the guidewire retainer 200, an opening 207 is formed for receipt of the guidewire. As part of its assembly for use in a medical procedure, the end of the guidewire may be inserted through the opening 207 and moved into the interior space. The guidewire end may be moved further into the interior space and stopped at or before a stop portion 208. Once the guidewire end stops, the portions 200A and 200B may be closed for securely attaching the guidewire retainer 200 to the end of the guidewire. In an example, the guidewire retainer 200 may be about 1 inch long or any other suitable length. Further, for example, the height and/or width of the guidewire retainer 200 may be about 0.25 inches or any other suitable height and width.

The closing of the grooves 202 and 204 upon the guidewire end can create a compressive force upon the guidewire end such that the guidewire end is resistant to forces pulling the guidewire out of the guidewire retainer 200. In an example, one or both of groove 202 and groove 204 may be formed of flexible material and/or shaped slightly smaller than the guidewire end to provide grip upon the guidewire end.

The guidewire retainer 200 may include a locking mechanism. The locking mechanism may include a notch 210 defined on the top portion 200B and an indention (not shown) formed at a corresponding position on the bottom portion 200B for attaching the top portion 200B and the bottom portion 200A in the closed position. This mechanism can securely hold the portions 200A and 200B together such that they attach the guidewire retainer 200 to the guidewire. In operation to attach the portions 200A and 200B, the portions 200A and 200B can be closed such that the notch 210 fits in the indention to attach the portions. The wireguide retainer 200 may also include a clip feature 212 that can “pinch” the guidewire to provide additional force for holding the guidewire end within the guidewire retainer 200.

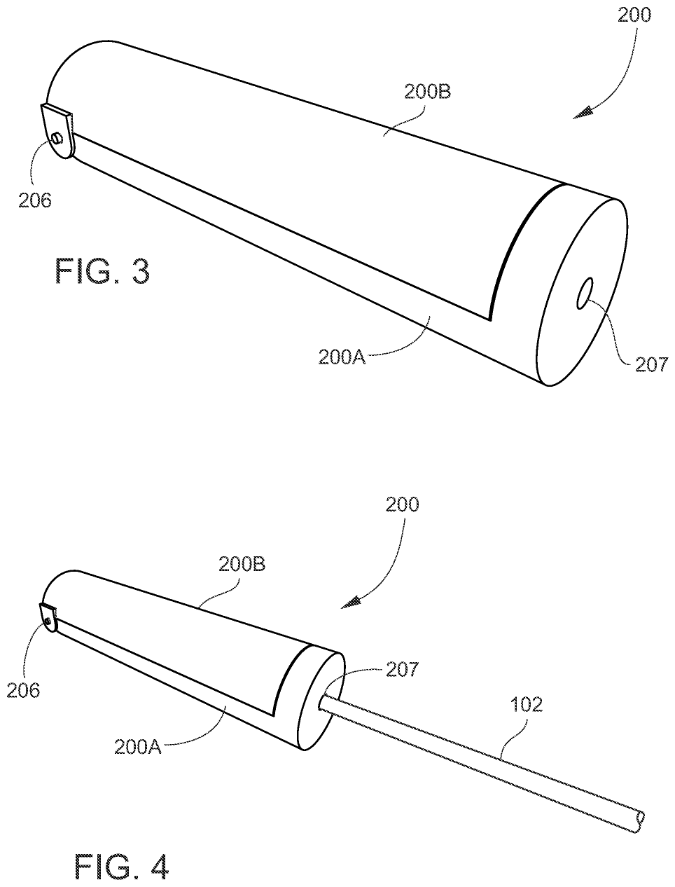

FIG. 3 illustrates a perspective view of the guidewire retainer 200 shown in FIG. 2 in a closed position. FIG. 4 illustrates a perspective view of the guidewire retainer 200 shown in FIG. 2 in a closed position with a guidewire 102 inserted therein.

In embodiments and examples provided herein, the guidewire retainer is described as being used with a catheter, although it should be appreciated that the guidewire retainer may alternatively be used with a guidewire and a dilator or any other tubular object.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. A guidewire retainer comprising:
a body that defines an interior space therein and an opening at one end that provides passage from an exterior of the body to the interior space, the interior space being shaped and sized for holding an end of a guidewire; and
a locking mechanism configured to close the body for attaching the body to the end of the guidewire.

2. The guidewire retainer of claim 1, wherein the body is one of a spherical shape, a conical shape, a cubical shape, a rectangular shape, a square shape, and a rhomboidal shape.

3. The guidewire retainer of claim 1, wherein the body comprises a first portion and a second portion,
wherein the locking mechanism is configured to attach the first portion and the second portion together.

4. The guidewire retainer of claim 3, wherein the first portion and the second portion define the interior space, and
wherein the first portion and the second portion are separable from one another such that the interior space is accessible.

5. The guidewire retainer of claim 3, wherein the locking mechanism locks the first and second portions in a closed position when the first and second portions are pressed together.

6. The guidewire retainer of claim 1, wherein the guidewire is a 20 gauge wire, has a length of about 1 centimeter to about 3 centimeters, and a width of about 0.1 centimeters to about 1 centimeter.

7. The guidewire retainer of claim 1, wherein the body has a length of about 1 centimeter to 2 centimeters, and a width of about 0.4 centimeters to about 0.6 centimeters.

8. A method of preventing a guidewire from completely entering a tubular object, wherein the method comprises:
providing a guidewire retainer comprising:
a body that defines an interior space therein and an opening at one end that provides passage from an exterior of the body to the interior space, the interior space being shaped and sized for holding an end of a guidewire; and
a locking mechanism configured to close the body for attaching the body to the end of the guidewire;
inserting the end of the guidewire into the interior space;
using the locking mechanism to attach the end of the guidewire to the body of the guidewire retainer.

9. The method of claim 8, wherein the body is one of a spherical shape, a conical shape, a cubical shape, a rectangular shape, a square shape, and a rhomboidal shape.

10. The method of claim 8, wherein the body comprises a first portion and a second portion,
wherein the locking mechanism is configured to attach the first portion and the second portion together.

11. The method of claim 10, wherein the first portion and the second portion define the interior space, and
wherein the first portion and the second portion are separable from one another such that the interior space is accessible.

12. The method of claim 10, wherein the locking mechanism locks the first and second portions in a closed position when the first and second portions are pressed together.

13. The method of claim 8, wherein the guidewire is a 20 gauge wire, has a length of about 1 centimeter to about 3 centimeters, and a width of about 0.1 centimeters to about 1 centimeter.

14. The method of claim 8, wherein the body has a length of about 1 centimeter to 2 centimeters, and a width of about 0.4 centimeters to about 0.6 centimeters.

15. The method of claim 8, further comprising inserting the guidewire into the tubular object.

16. The method of claim 15, further comprising:
inserting the guidewire through skin of a subject;
removing the needle; and
attaching the guidewire retainer to a proximal end of the guidewire to thereby prevent the guidewire from completely entering the tubular object.

17. The method according to claim 16, further comprising removing the guidewire retainer from the guidewire after completion of a medical procedure.

18. A kit comprising:
a tubular object or guidewire; and
a guidewire retainer comprising:
a body that defines an interior space therein and an opening at one end that provides passage from an exterior of the body to the interior space, the interior space being shaped and sized for holding an end of a guidewire; and
a locking mechanism configured to close the body for attaching the body to the end of the guidewire.

19. The kit of claim 18, wherein the tubular object is one of a catheter and dilator.

20. The kit of claim 18, wherein the body is one of a spherical shape, a conical shape, a cubical shape, a rectangular shape, a square shape, and a rhomboidal shape.

21. The kit of claim 18, wherein the body comprises a first portion and a second portion,
wherein the locking mechanism is configured to attach the first portion and the second portion together.

22. The kit of claim 21, wherein the first portion and the second portion define the interior space, and
wherein the first portion and the second portion are separable from one another such that the interior space is accessible.

23. The kit of claim 21, wherein the locking mechanism locks the first and second portions in a closed position when the first and second portions are pressed together.

24. The kit of claim 18, wherein the guidewire is a 20 gauge wire, has a length of about 1 centimeter to about 3 centimeters, and a width of about 0.1 centimeters to about 1 centimeter.

25. The kit of claim 18, wherein the body has a length of about 1 centimeter to 2 centimeters, and a width of about 0.4 centimeters to about 0.6 centimeters.
